(19) United States
(12) Patent Application Publication
(10) Pub. No.: US 2021/0077238 A1
(43) Pub. Date: Mar. 18, 2021

(54) Mouth Gag And Method For Using The Same

(71) Applicant: 3D Industrial Imaging Co., Ltd., Seoul (KR)

(72) Inventor: Kyu Nyun KIM, Seoul (KR)

(73) Assignee: 3D Industrial Imaging Co., Ltd., Seoul (KR)

(21) Appl. No.: 16/696,511

(22) Filed: Nov. 26, 2019

(30) Foreign Application Priority Data
Sep. 18, 2019 (KR) 10-2019-0114788

(51) Int. Cl. A61C 19/04; G01B 11/02

(52) U.S. Cl. CPC A61C 19/04; G01B 11/022

(57) ABSTRACT

Disclosed is a mouth gag, which comprises: a body placed in a patient's mouth to keep the mouth open and expose teeth; and a plurality of markers arranged on the body to allow for determining tooth size.

### CROSS REFERENCE TO RELATED APPLICATION

This application claims the benefit of and priority to Korean Patent Application No. 10-2019-0114788, filed on Sep. 18, 2019. The entire disclosure of the above application is incorporated herein by reference.

### FIELD

The present disclosure relates generally to a mouth gag and a method for using the same. More particularly, it relates to a mouth gag capable of measuring tooth size while tooth images are being taken, and a method for using the same.

### BACKGROUND

This section provides background information related to the present disclosure which is not necessarily prior art.

FIG. 1 shows an exemplary embodiment of a mouth gag described in Korean registered utility model gazette No. 20-0446039. A mouth gag 10, which is an aid for dental work or treatment, includes a body 11, an upper support 12, a lower support 14, and a water screen 13. The body 11 has an oval shape. The upper and lower supports 12 and 14 are inserted into their respective areas between the upper and lower lips and the upper and lower gums inside a patient's mouth, such that the upper and lower lips of the patient may be supported and remained open in the same shape as the oval body. The water screen 13 is adapted to prevent water droplets from splashing towards the philtrum area or getting into the nostrils of the patient when any dental instrument such as a handpiece is used.

In general, teeth are exposed through the mouth gag 10 as shown in FIG. 1, and images of those exposed teeth together with a ruler are then taken by a dentist or dental nurse. The ruler serves as a reference to determine the size of a tooth. However, when a patient takes a photo of his or her teeth exposed through the mouth gag 10 worn on the mouth and sends it to a dental office or dental laboratory, it is difficult to determine the actual sizes of the patient's teeth as there is no reference to compare with.

### DISCLOSURE

### Technical Problem

The problems to be solved by the present disclosure will be described in the latter part of the best mode for carrying out the invention.

### Technical Solution

This section provides a general summary of the disclosure and is not a comprehensive disclosure of its full scope or all of its features.

According to an aspect of the present disclosure, there is provided a mouth gag comprising: a body placed in a patient's mouth to keep the mouth open and expose teeth; and a plurality of markers arranged on the body to allow for determining tooth size.

According to another aspect of the present disclosure, there is provided a method for using a mouth gag, comprising: inserting a mouth gag provided with markers into a patient's mouth to expose teeth; photographing the exposed teeth together with the mouth gag; comparing shapes of the markers on a photographed image with shapes of the markers before photographing; and determining tooth size based on comparison results.

### Advantageous Effects

The advantageous effects of the present disclosure will be described in the latter part of the best mode for carrying out the invention.

### BRIEF DESCRIPTION OF THE DRAWINGS

FIG. 1 shows an exemplary embodiment of a mouth gag described in Korean registered utility model gazette No. 20-0446039.

FIG. 2 illustrates a mouth gag according to an exemplary embodiment of the present disclosure.

FIG. 3(A) and FIG. 3(B) illustrates a mouth gag according to another exemplary embodiment of the present disclosure.

FIGS. 4(A), 4(B), 4(C), 4(D), 4(E), 4(F) and FIG. 4(G) illustrates examples of a marker according to the present disclosure.

FIG. 5 is a flow chart describing a method for using a mouth gag according to the present disclosure.

FIG. 6 illustrates a mouth gag according to yet another exemplary embodiment of the present disclosure.

FIG. 7 is a view comparing the shapes of markers on a mouth gag according to the present disclosure, before and after photographing.

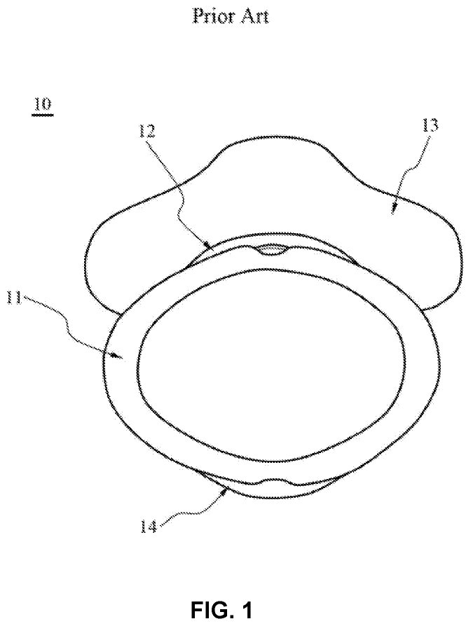

### DETAILED DESCRIPTION

Hereinafter, the present disclosure will now be described in detail with reference to the accompanying drawings.

In the description below, the phrase “at least one of A and B” should be understood to mean “only A, only B, or both A and B.”

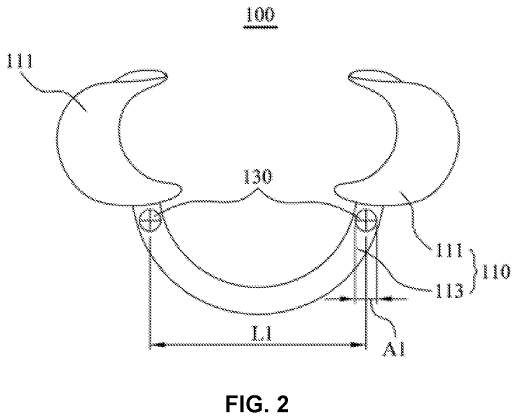

FIG. 2 illustrates a mouth gag 100 according to an exemplary embodiment of the present disclosure.

The mouth gag 100 includes a body 110 and markers 130. The body 110 includes a plurality of holders 111 and a connecting part 113.

The plurality of holders 111 is placed in a joining portion between the upper lip and the lower lip, and opens the mouth to expose teeth. The connecting part 113 connects the plurality of holders 111.

The markers 130 are arranged at the body 110. More than one marker is generally present. Tooth size can be determined using these markers 130. The term ‘tooth size’ may refer to height, width and area of a tooth.

The marker 130 can be at least one of a line segment having a predetermined length and a geometric shape having a predetermined area. The marker 130 may be an integrated part of the body 110, or may be a sticker to be attached to the body 110. The markers 130 may be arranged at a predetermined distance L from each other.

Once the mouth gag 100 in a folded state is placed into a mouth of a patient, it is fully spread apart. The predetermined distance L between the markers 130 is determined when the markers 130 are fully spread apart. These markers 130 feature at least one of a predetermined dimension A1 of each marker and the predetermined distance L defined as a physical length between centers of the markers. Both the predetermined distance L1 between the markers 130 and the predetermined dimension A1 of each maker are then used as references to determine at least one of lengths and widths of teeth being exposed. One example of the predetermined dimension A1 is a diameter.

As in FIG. 2, the marker 130 can be a circle with an equilateral cross inside, for example. A rotation degree of the marker 130 is taken with respect to the line segments of the cross. Each marker 130 has a diameter A1. The marker 130 can be used as a reference to determine tooth size. More specifically, the diameter A1 of the marker 130 can be used as a reference to determine the width, height and area of a tooth. Similarly, the predetermined distance L between the markers 130 can be used as a single reference to determine tooth size.

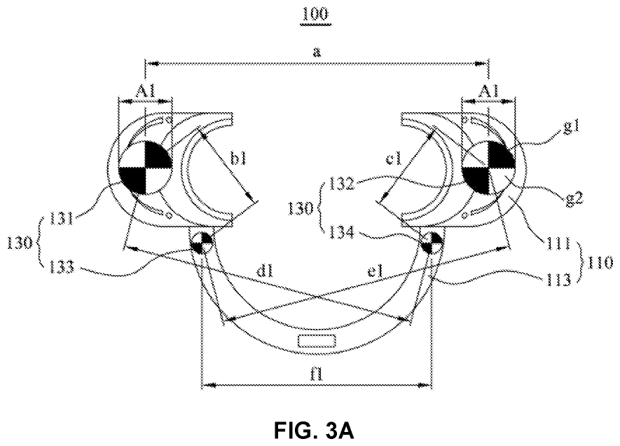

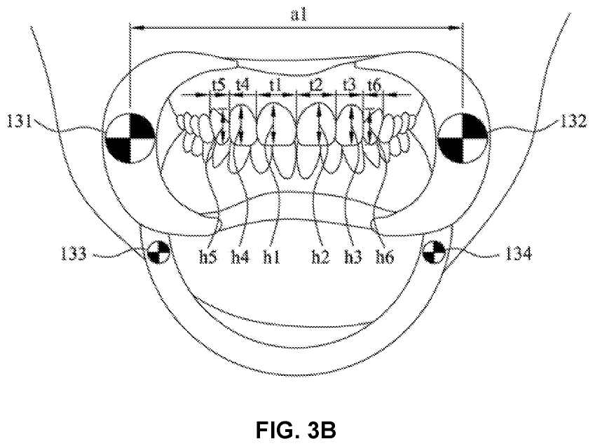

FIG. 3(A) and FIG. 3(B) illustrates a mouth gag according to another exemplary embodiment of the present disclosure.

The mouth gag has a plurality of markers 130 including a first marker 131, a second marker 132, a third marker 133 and a fourth marker 134. The first marker 131 and the second marker 132 may be formed at or attached to the holders 111, respectively. The third marker 133 and the fourth marker 134 may be formed at or attached to the connecting part 113. The first marker 131 and the second marker 132 are arranged at a predetermined distance from each other. Also, the third maker 133 and the fourth marker 134 are arranged at a predetermined distance from each other. The first marker 131 and the second marker 132 have a predetermined dimension. Also, the third maker 133 and the fourth marker 134 have a predetermined dimension.

By way of example, the first marker 131 and the second marker 132 each may have a diameter of 14 mm, and the third marker 133 and the fourth maker 134 each may have a diameter of 7 mm. Further, a distance al between the center of the first marker 131 and the center of the second marker 132 may be 8.5 cm, and a distance f1 between the center of the third marker 133 and the center of the fourth marker 134 may be 6.5 cm. Likewise, a distance b1 between the center of the first marker 131 and the center of the third marker 133, a distance dl between the center of the first marker 131 and the center of the fourth marker 134, a distance el between the center of the second marker 132 and the center of the third marker 133, and a distance c1 between the center of the second marker 132 and the center of the fourth marker 134 may be predetermined.

The first to fourth markers 131-134 have their own dimensions and are arranged at a predetermined distance from one another. At least one of the dimensions and the distances can be used for determining tooth size. Examples of the tooth size may include tooth height h1, h2, h3, h4, h5 or h6, tooth width t1, t2, t3, t4, t5 or t6, and area of each tooth.

Moreover, the marker 130 can have at least one color g1 and g2. These colors g1, g2 shown on the marker 130 are used to determine tooth color. For instance, the color shown on the marker 130 may be at least one of white and black. If it is white, it would correspond to A1 or B1 in a shade guide that is commonly used for in clinical dental practice. If it is black, it would correspond to the darkest achromatic color in the shade guide.

In addition, the marker 130 may have a line segment as a reference for the center and orientation of the marker 130.

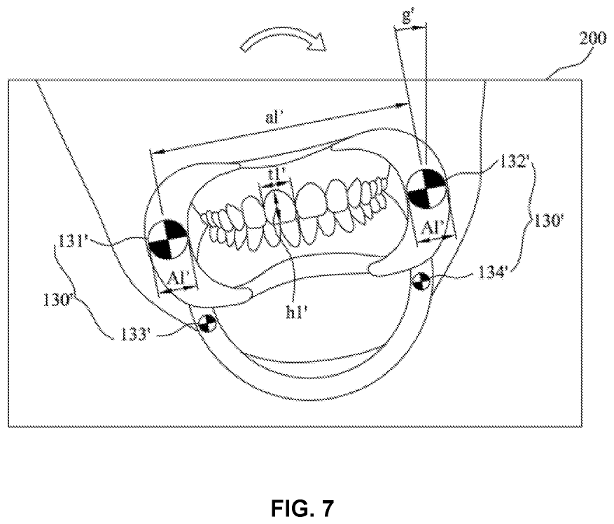

FIG. 3(B) illustrates how the mouth gag 100 of FIG. 3(A) looks when it is worn on a patient's mouth. This state shown in FIG. 3(B) is then photographed, as in FIG. 7.

Heights h1, h2, h3, h4, h5 and h6 as well as widths t1, t2, t3, t4, t5 and t6 of teeth can be determined based on the distance al between the first marker 131 and the second marker 132, and based on dimensions of the first to fourth markers 131-134.

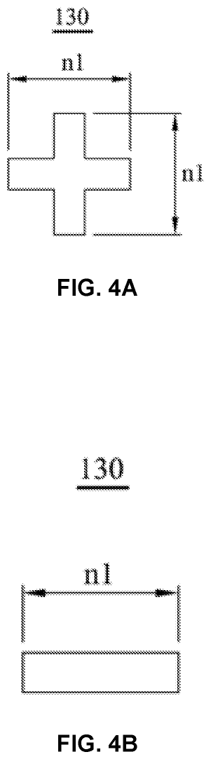

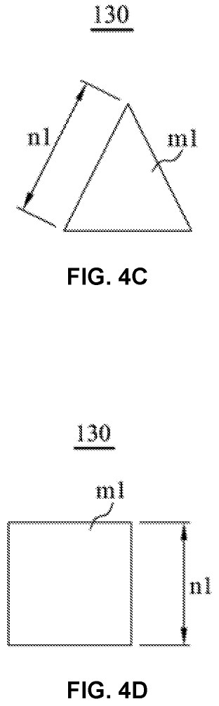

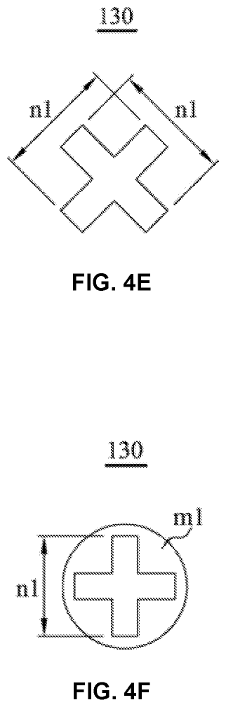

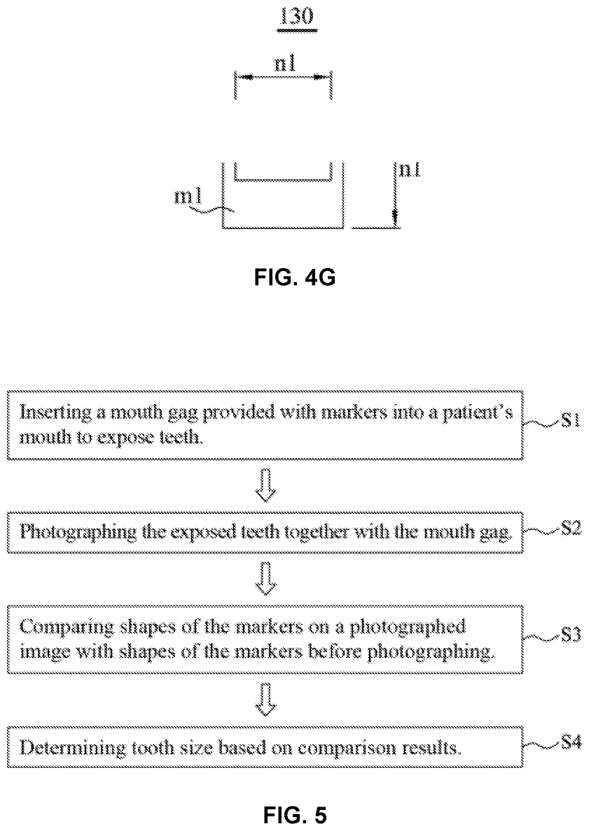

FIG. 4(A) to FIG. 4(G) illustrates different examples of the marker 130 according to the present disclosure.

The marker 130 can be formed of line segments such as an equilateral cross, a horizontal line segment or a vertical line segment, or a variety of geometric shapes. When the marker 130 is a line segment, it can have a predetermined length n1. When the marker 130 is a geometric shape, it can have a predetermined area ml, as well as a predetermined length n1.

In addition, the marker 130 may have line segments such as an equilateral cross, a horizontal line segment or a vertical line segment inside a shape. Although not shown, the marker 130 may optionally have colors.

FIG. 5 is a flow chart describing a method for using the mouth gag 100 according to the present disclosure.

According to the method for using the mouth gag 100, first, the mouth gag 100 having predetermined markers 130 is inserted into a patient's mouth to expose teeth (S1).

An image of those exposed teeth, together with the mouth gag 100, is then taken (S2). This is possible as the markers 130 are arranged on the mouth gag 100 in the same direction as the exposed teeth. Preferably, the teeth and the markers are photographed.

The shape of marker 130′ (see FIG. 7) on the mouth gag 100 shown in the photograph is compared with the shape of the marker 130 on the mouth gag 100 before photographing (S3). That is, the shape of the marker 130′ (see FIG. 7) image after photographing is compared with the shape of the marker 130 before photographing. The marker 130 before photographing can be a reference marker image, which is an original image with an actual dimension A1 of the marker 130.

The shape of the marker 130′ on the photographed image is calibrated to be identical with the shape of the marker 130 on the original image. During this calibration operation for matching the marker 130′ on the photographed image with the marker 130, an entire photographed image 200 (see FIG. 7) can be calibrated at the same time. For example, a computer program may run to determine a calibrated tooth size with reference to the marker 130′ on the photographed image 200.

When the marker 130 has a line segment, comparison between the shape of the marker 130′ on the photographed image and the shape of the marker 130 before photographing can be accomplished by comparing at least one of contrast, orientation or length of the line segment in both markers.

When the marker 130 is a geometric shape having a certain area, comparison between the shape of the marker 130′ on the photographed image and the shape of the marker 130 before photographing can be accomplished by comparing at least one of contrast, orientation or area of the line segment in both markers.

The distance al (see FIG. 3) between the markers 130 can be another reference for calibrating the marker 130′ on the photographed image.

Finally, tooth size is determined based on comparison results (S4). More specifically, tooth size is determined based on at least one of a dimension A1 of the marker 130 and a distance between the markers 130. Examples of the dimension A1 of the marker 130 may include length, area and geometric shape.

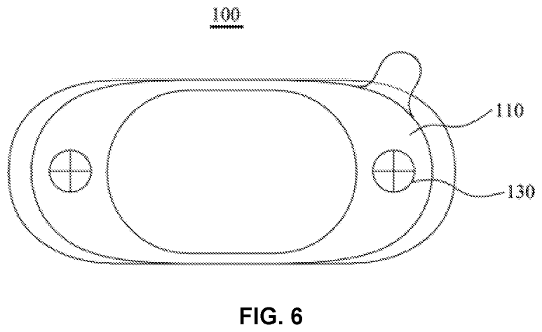

FIG. 6 illustrates a mouth gag 100 according to yet another exemplary embodiment of the present disclosure.

The mouth gag 100 includes a body 110 and markers 130. The body 110 is arranged between the gums and lips of a patient to make the mouth open. The body 110 is inserted into a joining portion between the upper lip and the lower lip, forming a closed loop as seen from outside. The body 110 is not particularly limited to certain shapes illustrated in FIG. 2 to FIG. 6, as long as it is adapted to open the mouth and expose teeth. The markers 130 are preferably arranged on the side of the body 110 in the direction to expose the teeth of a patient wearing the body 110 with his or her mouth agape.

FIG. 7 is a view describing a step of comparing the shapes of markers on a mouth gag according to the present disclosure, before and after photographing.

A photographed image 200 of the mouth gag 100 worn on a patient's mouth is provided in FIG. 7. On the other hand, the mouth gag 100 (see FIG. 2) before photographing is provided in FIG. 3(A).

Compared with the markers 130 in FIG. 3(A) as a reference, the markers 130′ in FIG. 7 are rotated and different in size. Therefore, it is necessary to calibrate a distance al′ between the markers 130′ in FIG. 7 to the distance al between the markers 130 in FIG. 3(A), to calibrate a dimension A1′ of the marker 130′ in FIG. 7 to the dimension A1 of the marker 130 in FIG. 3(A), or to calibrate the cross of the marker 130′ in FIG. 7 to the cross of the marker 130 in FIG. 3(A). This process makes it possible to find out how much the marker 130′ on the photographed image has been diminished/magnified, or how much the marker 130′ on the photographed image has been rotated, with respect to the marker 130 before photographing. It involves rotating the marker 130′ in a rotating direction and angle g′ known during calibration. Once the photographed image underwent size calibration, i.e. the marker 130′ on the photographed image is either magnified or diminished such that the dimension A1′ of the marker 130′ on the photographed image is identical with the dimension A1 of the marker 130 in FIG. 3(A), tooth size (e.g., t1′, h1′) can be determined.

The photographed image 200 of the mouth gag 100 worn on the patient's mouth may undergo calibration with reference to the first and second markers 131 and 132 having a diameter A1 of 14 mm. For instance, if it turns out that first and second markers 131′ and 132′ on the photographed image 200 of the mouth gag 100 worn on the patient's mouth have a diameter A1′ of 7 mm, the photographed image 200 is then magnified two times such that the diameter A1′ on the photographed image 200 reaches 14 mm which is the diameter A1 of the first and second markers 131 and 132 before photographing. Following this calibration, it is possible to determine tooth size including, for example, height h1′ and width t1′ of a tooth exposed through the mouth gag 100.

It should be noted that image calibration described above is used for better understanding. In practice, the calibration process is carried out by a program such that tooth size including height h1′ or width t1′ of a tooth on the photographed image can still be obtained in absence of the calibrated photographed image.

While the above example has used the diameter A1′ of the marker 130′ as a reference to determine the actual size of a tooth of interest, the distance al between the markers 130, dimensions of the markers 130, or crosses of the markers 130 may also serve as references for the same purpose.

That is to say, a reference for tooth size may be any one selected from the distance al between the markers 130, dimensions of the markers 130, and crosses of the markers 130. Optionally, a reference for tooth size may be a combination of at least two selected from the distance al between the markers 130, dimensions of the markers 130, and crosses of the markers 130.

Further, the dimension A1′ of the first marker 131′ may be compared with the dimension A1′ of the second marker 132′. This comparison between the dimensions A′ of the first and second markers 131′ arranged at either side of the face on the photographed image 200 allows for determining how much the patient's face is rotated laterally. Likewise, the cross of the second marker 132′ can be compared with the cross of the second marker 132 so as to determine how much the photographed image 200 is rotated.

Set out below are clauses that describe diverse features of further aspects of the present disclosure.

(1) A mouth gag comprising: a body placed in a patient's mouth to keep the mouth open and expose teeth; and a plurality of markers arranged on the body to determine tooth size.

(2) There is also provided, the mouth gag of clause (1) wherein the marker is a line segment having a predetermined length.

(3) There is also provided, the mouth gag of clause (1) wherein the marker is a geometric shape having a predetermined area.

(4) There is also provided, the mouth gag of clause (3) wherein the marker has at least two colors.

(5) There is also provided, the mouth gag of clause (4) wherein tooth colors are compared with reference to the colors of the markers.

(6) There is also provided, the mouth gag of clause (1) wherein the plurality of markers has a predetermined distance between centers.

(7) There is also provided, the mouth gag of clause (1) wherein the marker is a sticker.

(8) There is also provided, the mouth gag of clause (1) wherein the body is inserted into a joining portion between the upper lip and the lower lip, forming a closed loop.

(9) There is also provided, the mouth gag of clause (1) wherein the body comprises: a plurality of holders placed in the joining portion between the upper lip and the lower lip; and a connecting part for connecting the plurality of holders.

(10) A method for using a mouth gag, comprising: inserting a mouth gag provided with markers into a patient's mouth to expose teeth; photographing the exposed teeth together with the mouth gag; comparing shapes of the markers on a photographed image with shapes of the markers before photographing; and determining tooth size based on comparison results.

(11) There is also provided, the method for using a mouth gag of clause (10) wherein comparing shapes of the markers on a photographed image with shapes of the markers before photographing includes comparing at least one of contrast, orientation or length, if the markers are line segments having a predetermined length.

(12) There is also provided, the method for using a mouth gag of clause (10) wherein comparing shapes of the markers on a photographed image with shapes of the markers before photographing includes comparing at least one of contrast, orientation or area, if the markers are geometric shapes having a predetermined area.

(13) There is also provided, the method for using a mouth gag of clause (10) wherein, in comparing shapes of the markers on a photographed image with shapes of the markers before photographing, the markers are arranged in a direction where the teeth are exposed.

(14) There is also provided, the method for using a mouth gag of clause (10) further comprising: after comparing shapes of the markers on a photographed image with shapes of the markers before photographing, measuring a distance between the markers.

A mouth gag according to an exemplary embodiment according to the present disclosure allows for determining tooth size with reference to markers arranged on the mouth gag.

A mouth gag according to another exemplary embodiment of the present disclosure allows for determining tooth size through a photographed image of the mouth gag and markers.

A method for using a mouth gag according to an exemplary embodiment of the present disclosure allows a patient (or user) to photograph an image of the mouth gag worn on his or her mouth, and to send the photographed image to a dental laboratory to make laminates, dentures, orthodontic appliances or prostheses.

A method for using a mouth gag according to another exemplary embodiment of the present disclosure is accomplished by a dentist who can utilize a photographed image of the mouth gag as a reference to determine tooth size.

Description of Reference Numerals

| | |
|---|---|
| 100: Mouth gag | 110: Body |
| 111: Holder | 113: Connecting part |
| 130: Marker | 131: First marker |
| 132: Second marker | 133: Third marker |
| 134: Fourth marker | |

What is claimed is:

1. A mouth gag comprising:
a body placed in a patient's mouth to keep the mouth open and expose teeth; and
a plurality of markers arranged on the body to allow for determining tooth size.

2. The mouth gag according to claim 1, wherein the marker is a line segment having a predetermined length.

3. The mouth gag according to claim 1, wherein the marker is a geometric shape having a predetermined area.

4. The mouth gag according to claim 3, wherein the marker has at least two colors.

5. The mouth gag according to claim 4, wherein tooth colors are compared with reference to the colors of the markers.

6. The mouth gag according to claim 1, wherein the plurality of markers has a predetermined distance between centers.

7. The mouth gag according to claim 1, wherein the marker is a sticker.

8. The mouth gag of according to claim 1, wherein the body is inserted into a joining portion between the upper lip and the lower lip, forming a closed loop.

9. The mouth gag according to claim 1, wherein the body comprises:
a plurality of holders placed in the joining portion between the upper lip and the lower lip; and
a connecting part for connecting the plurality of holders.

10. A method for using a mouth gag, comprising:
inserting a mouth gag provided with markers into a patient's mouth to expose teeth;
photographing the exposed teeth together with the mouth gag;
comparing shapes of the markers on a photographed image with shapes of the markers before photographing; and
determining tooth size based on comparison results.

11. The method for using a mouth gag according to claim 10, wherein comparing shapes of the markers on a photographed image with shapes of the markers before photographing includes comparing at least one of contrast, orientation or length, if the markers are line segments having a predetermined length.

12. The method for using a mouth gag according to claim 10, wherein comparing shapes of the markers on a photographed image with shapes of the markers before photographing includes comparing at least one of contrast, orientation or area, if the markers are geometric shapes having a predetermined area.

13. The method for using a mouth gag according to claim 10, wherein, in comparing shapes of the markers on a photographed image with shapes of the markers before photographing, the markers are arranged in a direction where the teeth are exposed.

14. The method for using a mouth gag according to claim 10, further comprising:
after comparing shapes of the markers on a photographed image with shapes of the markers before photographing, measuring a distance between the markers.
